(19) United States
(12) Patent Application Publication
(10) Pub. No.: US 2011/0286885 A1
(43) Pub. Date: Nov. 24, 2011

(54) MICROFLUIDIC DEVICE HAVING NORMALLY OPEN TYPE MICROVALVE AND METHOD OF MANUFACTURING THE MICROFLUIDIC DEVICE

(75) Inventors: Chin-sung PARK, Yongin-si (KR); Won-jong JUNG, Seongnam-si (KR); Jae-young KIM, Seoul (KR); Joon-ho KIM, Seongnam-si (KR)

(73) Assignee: SAMSUNG ELECTRONICS CO., LTD., Suwon-si (KR)

(21) Appl. No.: 12/959,719

(22) Filed: Dec. 3, 2010

(30) Foreign Application Priority Data
May 18, 2010 (KR) 10-2010-0046592

(51) Int. Cl. G01N 33/00; B44C 1/22

(52) U.S. Cl. 422/68.1; 216/33

(57) ABSTRACT

A microfluidic device includes a microvalve formed by disposing a thin elastic film and a valve seat in a microfluidic channel of the microfluidic device. The microvalve is a normally open type valve in which the elastic film does not contact the valve seat. According to the microvalve of the microfluidic device, an additional process for separating the elastic film from the valve seat is not required. Accordingly, the microfluidic device may have a relatively simple manufacturing process. Also, since an initialization operation to use the microfluidic device is not required, the flow of a fluid in the microfluidic device may be efficiently controlled without additional processing steps.

### CROSS-REFERENCE TO RELATED APPLICATION

This application claims priority to Korean Patent Application No. 10-2010-0046592, filed on May 18, 2010, and all the benefits accruing therefrom under 35 U.S.C. §119, the content of which in its entirety is herein incorporated by reference.

### BACKGROUND

1. Field

The present disclosure relates to microfluidic devices having a normally open type microvalve and methods of manufacturing the microfluidic devices, and more particularly, to microfluidic devices having a normally open type microvalve, in which an elastic film and a valve seat of the microvalve do not normally contact each other, and methods of manufacturing the microfluidic devices.

2. Description of the Related Art

A clinical or environment-related sample may be analyzed by performing a series of biochemical, chemical, and mechanical processes. Currently, development of technologies for diagnosing or monitoring a biological sample are attracting increased attention. Due to its excellent accuracy and sensitivity, a molecular diagnosis method based on a nucleic acid is increasingly and broadly being used to diagnose infectious diseases and cancers, to study pharmacogenomics, and to develop new medicines. Microfluidic devices are commonly used to analyze a sample simply and precisely for the various purposes described above. In a microfluidic device, a plurality of sample inlets, sample outlets, microfluidic channels, reaction chambers, etc., are formed on a thin substrate and thus various tests may be simply performed on one sample.

Microvalves may be provided in the microfluidic channels in order to accurately direct a sample or a reagent to a desired location in the microfluidic device. For example, a microvalve may typically be formed by disposing a thin elastic film and a valve seat in a microfluidic channel of the microfluidic device. In general, the microvalve is closed while the elastic film contacts the valve seat to prevent a sample from flowing through the microfluidic channel, and is open when the elastic film does not contact the valve seat to allow the sample to flow through the microfluidic channel.

### SUMMARY

Provided herein are easily manufacturable microfluidic devices having a normally open type microvalve, capable of efficiently controlling the flow of a fluid.

Provided herein are methods of manufacturing the microfluidic devices.

Additional aspects will be set forth in part in the description which follows and, in part, will be apparent from the description, or may be learned by practice of the presented embodiments.

According to an aspect of the present disclosure, a microfluidic device includes; a first substrate, and a second substrate disposed substantially opposite to the first substrate, an elastic film interposed between the first and second substrates, a microfluidic channel formed in a first surface of the second substrate, wherein the first surface of the second substrate faces the first substrate, a valve seat in which protrudes from the microfluidic channel of the second substrate, and an empty space formed in a first surface of the first substrate, wherein the first surface of the first substrate faces the second substrate and wherein the empty space corresponds to the valve seat, wherein an upper surface of the valve seat contacts the elastic film when a controlling air pressure is provided into the empty space, and a gap is formed between the upper surface of the valve seat and the elastic film when a controlling air pressure is not provided into the empty space.

For example, in one embodiment, a default setting of the elastic film and the valve seat may form a normally open-type microvalve.

In one embodiment, the microfluidic channel may be recessed in the first surface of the second substrate.

In one embodiment, the upper surface of the valve seat formed in the recessed microfluidic channel may be lower than the first surface of the second substrate.

For example, in one embodiment the elastic film may comprise polydimethylsiloxane.

In one embodiment, the first substrate and the second substrate include at least one of glass and plastic.

In one embodiment, the microfluidic device may further include a plurality of reaction chambers formed in at least one of the first surface of the first substrate and the first surface of the second substrate.

Also, the microfluidic device may further include a first hole formed in a second surface of the first substrate which is substantially opposite to the first surface of the first substrate, wherein the first hole is connected to the empty space.

Furthermore, the microfluidic device may further include a second hole formed in a second surface of the second substrate which is substantially opposite to the first surface of the second substrate, wherein the first hole is connected to the microfluidic channel.

In one embodiment, the microfluidic device may further include a microfluidic channel formed in the first surface of the first substrate.

In one embodiment, the gap between the upper surface of the valve seat and the elastic film while an air pressure is not provided into the empty space may be, for example, 0 μm to 20 μm.

According to another aspect of the present disclosure, a method of manufacturing a microfluidic device includes; wet etching a first surface of a first substrate to form an empty space in the first surface of the first substrate, wet etching a first surface of a second substrate to form a microfluidic channel and a valve seat which protrudes from the microfluidic channel in the first surface of the second substrate, interposing an elastic film between the first and second substrates while the first surface of the first substrate and the first surface of the second substrate face each other, and bonding the elastic film to the first substrate and the second substrate, wherein the empty space of the first substrate corresponds to the valve seat of the second substrate, and wherein an upper surface of the valve seat contacts the elastic film when a controlling air pressure is provided into the empty space, and a gap is formed between the upper surface of the valve seat and the elastic film when a controlling air pressure is not provided into the empty space.

Also, the wet etching a first surface of the second substrate to form the valve seat may include; sequentially coating an etching mask and a photoresist on the first surface of the second substrate; patterning the etching mask and the photoresist until only a portion of the etching mask remains; and partially etching the first surface of the second substrate such that an upper surface of the valve seat is lower than the first surface of the second substrate.

In one embodiment, the patterning of the etching mask and the photoresist may include patterning the photoresist by exposing and developing the photoresist; and patterning the etching mask by removing portions of the etching mask from which the photoresist is removed, using a deep reactive ion etching (“DRIE”) method.

For example, in one embodiment, when a width of the portion of the etching mask for forming the valve seat is W_{ETCH} and an etching depth of the first surface of the second substrate is D, the following inequality may be satisfied; W_{ETCH}≦2×D.

In one embodiment, a height of the upper surface of the valve seat may be lower than a height of the first surface of the second substrate by, for example, about 0 μm to about 20 μm.

Also, in one embodiment, the interposing of the elastic film between the first and second substrates and the bonding of the elastic film to the first substrate and the second substrate may include interposing the elastic film between the first surface of the first substrate and the first surface of the second substrate, processing the first and second substrates and the elastic film using oxygen (O₂) plasma, and heating the first and second substrates and the elastic film in an oven.

### BRIEF DESCRIPTION OF THE DRAWINGS

These and/or other aspects will become apparent and more readily appreciated from the following description of the embodiments, taken in conjunction with the accompanying drawings of which:

FIG. 1 is a top plan view of an embodiment of a microfluidic device according to the present disclosure;

FIG. 2 is a cross-sectional view of the embodiment of a microfluidic device illustrated in FIG. 1;

FIG. 3 is a top plan view of one of the embodiments of a microvalve formed in the microfluidic device illustrated in FIG. 1, according to the present disclosure;

FIG. 4A is a cross-sectional view of the embodiment of a microvalve illustrated in FIG. 3 along line A-A′ of FIG. 3;

FIG. 4B is a cross-sectional view of the embodiment of a microvalve illustrated in FIG. 3 along line B-B′ of FIG. 3;

FIG. 5A is a cross-sectional view showing an opening operation of the embodiment of a microvalve illustrated in FIG. 4A;

FIG. 5B is a cross-sectional view showing a closing operation of the embodiment of a microvalve illustrated in FIG. 4A;

FIG. 5C is a cross-sectional view showing a closing operation of the embodiment of a microvalve illustrated in FIG. 4B; and

FIGS. 6A through 6D are cross-sectional views illustrating an embodiment of a method of manufacturing the microvalve illustrated in FIGS. 3, 4A, and 4B, according to the present disclosure.

### DETAILED DESCRIPTION

Embodiments now will be described more fully hereinafter with reference to the accompanying drawings, in which embodiments are shown. These embodiments may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present disclosure.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another elements as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments should not be construed as limited to the particular shapes of regions illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the disclosure.

All methods described herein can be performed in a suitable order unless otherwise indicated herein or otherwise clearly contradicted by context. The use of any and all examples, or exemplary language (e.g., “such as”), is intended merely to better illustrate the disclosure and does not pose a limitation on the scope thereof unless otherwise claimed. No language in the specification should be construed as indicating any non-claimed element as essential to the practice of the embodiments as used herein.

Hereinafter, the embodiments will be described in detail with reference to the accompanying drawings.

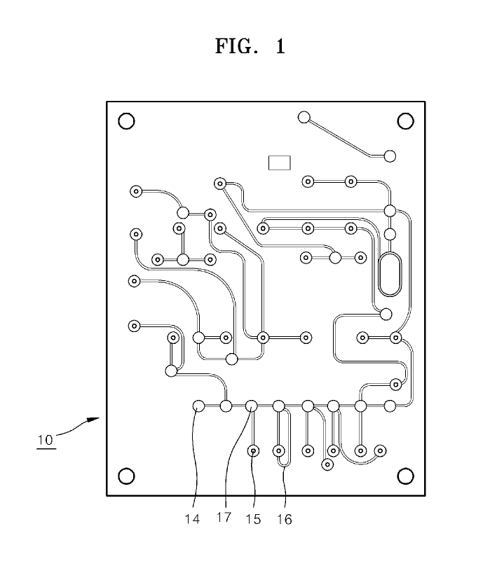

FIG. 1 is a top plan view of an embodiment of a microfluidic device 10 according to the present disclosure. Referring to FIG. 1, the microfluidic device 10 may include a plurality of holes 15 formed in a thin and transparent substrate to accommodate the inflow or outflow a sample, a reagent, or air, a plurality of reaction chambers 14 in which chemical/biological reaction involving the sample occurs, a plurality of microfluidic channels 16 through which the sample flows, and a plurality of microvalves 17 for accurately controlling the flow of the sample to a desired location. In FIG. 1, although reference numerals are representatively marked on one reaction chamber 14, one hole 15, one microfluidic channel 16, and one microvalve 17 for convenience, a plurality of reaction chambers 14, holes 15, microfluidic channels 16, and microvalves 17 are illustrated. Also, in FIG. 1, the numbers and arrangements of the reaction chambers 14, the holes 15, the microfluidic channels 16, and the microvalves 17 of the microfluidic device 10 are exemplarily illustrated, and may vary according to the use and particular configuration of the microfluidic device 10.

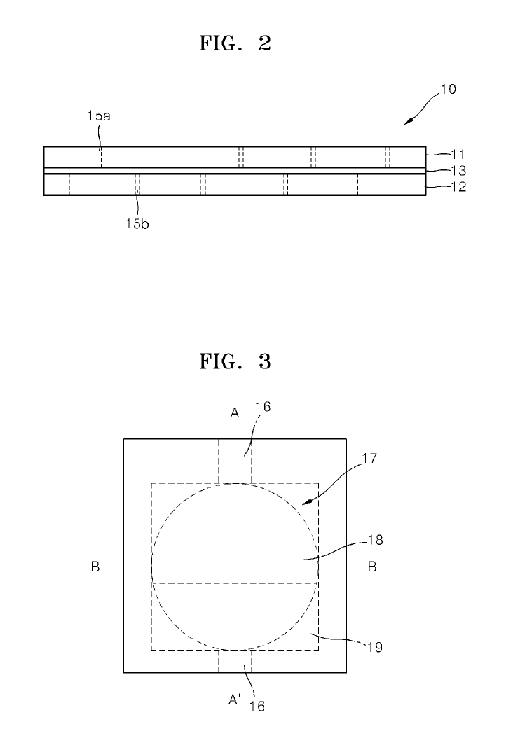

The microvalves 17 may be formed in the microfluidic channels 16 and pass or block the sample as the sample flows through the microfluidic channels 16. In one embodiment, the microvalves 17 may be formed of an elastic thin film. FIG. 2 is a cross-sectional view of the microfluidic device 10 illustrated in FIG. 1. Referring to FIG. 2, the microfluidic device 10 may include first and second substrates 11 and 12, and a comparatively thin elastic film 13 formed between the first and second substrates 11 and 12. A plurality of first holes 15a may be formed in the first substrate 11 and a plurality of second holes 15b may be formed in the second substrate 12. As will be described in more detail later, the first holes 15a may be air pressure holes for providing an air pressure to push the elastic film 13 and the second holes 15b may be fluid holes for providing a fluid, such as the sample; the air pressure may also be referred to herein as a controlling air pressure because the air pressure applied through the holes is used to control the configuration of the elastic film 13, and the air pressure may be different than an ambient air pressure surrounding the microfluidic device. Although only the first and second holes 15a and 15b are illustrated in FIG. 2 for convenience, the pluralities of reaction chambers 14 and microfluidic channels 16 may be individually formed in opposing surfaces of the first and second substrates 11 and 12. In the present embodiment, the elastic film 13 may be formed of a polymer material such as polydimethylsiloxane (“PDMS”). In one embodiment, the first and second substrates 11 and 12 may be formed of a transparent material such as glass, plastic or other material with similar characteristics.

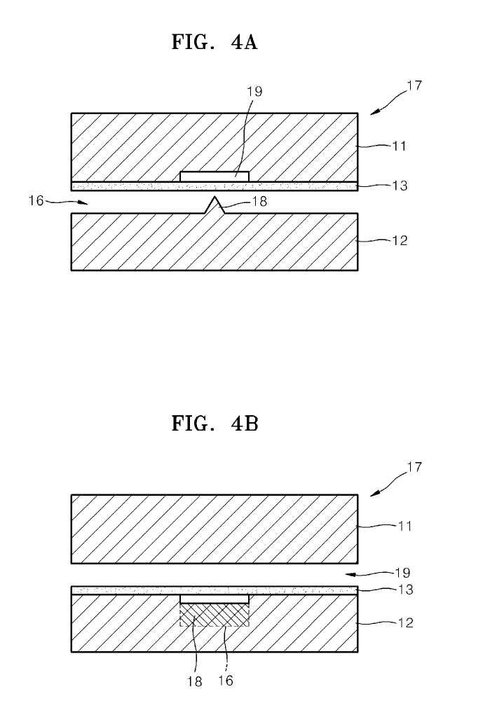

FIG. 3 is a top plan view of one of the microvalves 17 formed in the embodiment of a microfluidic device 10 illustrated in FIG. 1, according to the present disclosure. In FIG. 3, the structure of the microvalve 17 is illustrated by using dashed lines. Referring to FIG. 3, a valve seat 18 is formed to cross the microfluidic channel 16. The width of the microfluidic channel 16 may be slightly increased where the valve seat 18 is formed, in order to allow the microvalve 17 to easily operate. For example, the microfluidic channel 16 and the valve seat 18 may be formed contiguously in the second substrate 12. Although not shown in FIG. 3, the elastic film 13 is formed over the valve seat 18. An empty space 19 may be formed in a region of the first substrate 11 disposed on the elastic film 13 and corresponding to the valve seat 18. The empty space 19 is used to provide a controlling air pressure to push the elastic film 13 toward the valve seat 18 with a desired strength during a closing operation of the microvalve 17, as will be discussed in more detail below.

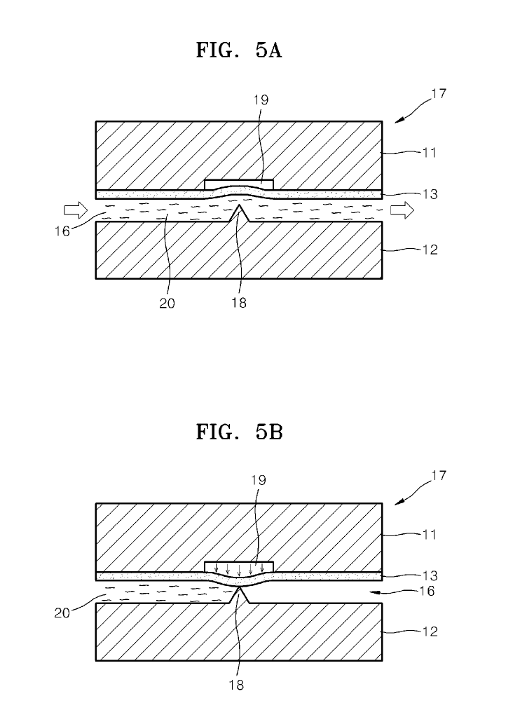

The structure of the microvalve 17 is illustrated in detail in FIGS. 4A and 4B. FIG. 4A is a cross-sectional view of the microvalve 17 illustrated in FIG. 3 taken along line A-A′ of FIG. 3. FIG. 4B is a cross-sectional view of the microvalve 17 illustrated in FIG. 3 taken along line B-B′ of FIG. 3.

Referring to FIG. 4A, the valve seat 18 protrudes from a bottom surface of the microfluidic channel 16 formed in an upper surface of the second substrate 12 facing the first substrate 11. Also, the empty space 19 is formed in a region of a lower surface of the first substrate 11 facing the second substrate 12 and corresponding to the valve seat 18, e.g., by etching the lower surface of the first substrate 11. The empty space 19 and the valve seat 18 are separated from each other by the elastic film 13 between the first and second substrates 11 and 12. The elastic film 13 is bonded to the lower surface of the first substrate 11. An upper surface of the valve seat 18 is adjacent to the elastic film 13 but, in a default state, does not contact the elastic film 13. Meanwhile, although not shown in FIGS. 4A-B, the microfluidic channel 16 may be connected to the second holes 15b formed in the second substrate 12 to inflow/outflow the sample, and the empty space 19 may be connected to the first holes 15a formed in the first substrate 11 to inflow/outflow air.

Also, referring to FIG. 4B, the empty space 19 is formed in the first substrate 11 and the valve seat 18 is formed in the second substrate 12. The microfluidic channel 16 (represented using a dashed line in FIG. 4B) is connected to front and back sides of the valve seat 18; as used herein, the term “front” describes a portion of the valve seat 18 closest to the microfluidic channel 16 from which a fluid flows into the microvalve 17 and the term “back” describes a portion of the valve seat 18 closest to the microfluidic channel 16 to which a fluid flows out of the microvalve 17. In FIG. 4B, the upper surface of the second substrate 12 is bonded to the elastic film 13. As described above in relation to FIG. 4A, in a default state the upper surface of the valve seat 18 does not contact the elastic film 13. As illustrated in FIG. 4B, the height of the upper surface of the valve seat 18 may be lower than the height of the upper surface of the second substrate 12. Thus, the upper surface of the valve seat 18 does not contact the elastic film 13 and a gap is formed between the upper surface of the valve seat 18 and the elastic film 13. For example, the gap between the upper surface of the valve seat 18 and the elastic film 13 may be about 0 μm to about 20 μm.

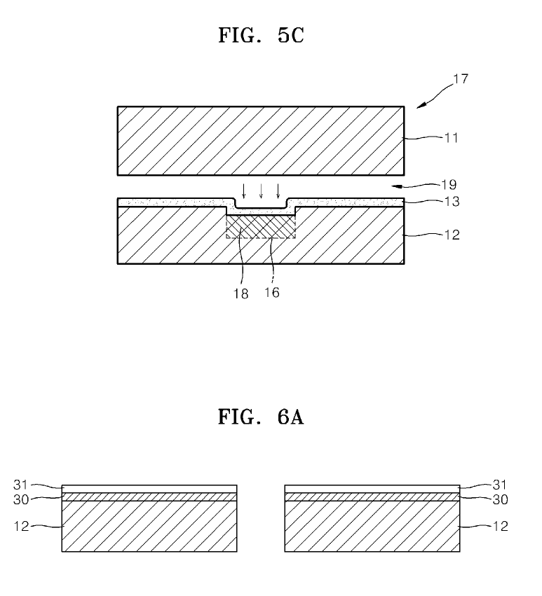

Opening and closing operations of the microvalve 17 will now be described with reference to FIGS. 5A and 5B. FIG. 5A is a cross-sectional view showing an opening operation of the embodiment of a microvalve 17 illustrated in FIG. 4A. FIG. 5B is a cross-sectional view showing a closing operation of the embodiment of a microvalve 17 illustrated in FIG. 4A. FIG. 5C is a cross-sectional view showing a closing operation of the embodiment of a microvalve 17 illustrated in FIG. 4B. Referring to FIG. 5A, since the upper surface of the valve seat 18 does not contact the elastic film 13 as described above in relation to FIGS. 4A and 4B, the microvalve 17 is a normally open type, e.g., the default state of the microvalve 17 is an open state. Accordingly, the microvalve 17 is normally open and thus a fluid 20 provided into the microfluidic channel 16 via the second holes 15b may pass through the microvalve 17.

In order to close the microvalve 17, as illustrated in FIGS. 5B and 5C, a controlling air pressure is provided into the empty space 19 via the first holes 15a. The controlling air pressure is of a sufficient pressure to deform the elastic film 13 as described below, and as such may be greater than the atmospheric pressure surrounding the microfluidic device. As such, the elastic film 13 under the empty space 19 is pressed toward the valve seat 18 by the applied controlling air pressure. If the air pressure is provided at a sufficient pressure, the elastic film 13 may contact the valve seat 18 and fill the gap between the elastic film 13 and the valve seat 18. As such, the fluid 20 in the microfluidic channel 16 may be blocked by the microvalve 17 and may not flow through the microvalve 17. Here, the applied pressure of the controlling air pressure is influenced by various factors such as the material used to form the elastic film 13, the distance between the valve seat 18 and the elastic film 13, the width and height of the microfluidic channel 16, and surface states and geometrical shapes of the valve seat 18 and the elastic film 13, and thus will not be specifically defined here.

Forming of the microvalve 17 as a normally open type as described above may have various advantages. Initially, when the elastic film 13 is disposed between the first and second substrates 11 and 12 as illustrated in FIG. 2, the elastic film 13 may be bonded to be fixed on the first and second substrates 11 and 12. However, it is not desirable that the elastic film 13 is bonded to the valve seat 18. Accordingly, in contrast to the microvalve 17 described above, in a normally closed type in which the valve seat 18 normally contacts the elastic film 13, the surface of the valve seat 18 may require an additional coating so that the valve seat 18 is not bonded to the elastic film 13. As such, a microfluidic device having a normally closed type microvalve has a relatively complicated manufacturing process and the bonding strength between the elastic film 13 and the first and second substrates 11 and 12 may be relatively weakened by the additional coating process. Also, after the conventional microfluidic device is completely manufactured, in order to ensure proper separation between the valve seat 18 and the elastic film 13, a relatively high air pressure may be required to be provided through the microfluidic channel 16, which may cause a gap between the first and second substrates 11 and 12 and the elastic film 13 and thus the fluid 20 may leak. In contrast, in the disclosed microvalve 17, since the valve seat 18 does not normally contact the elastic film 13, the above problem may be prevented. Therefore, the microfluidic device 10 may have a relatively simple manufacturing process. Also, since the elastic film 13 may be strongly bonded to the first and second substrates 11 and 12 without being bonded to the valve seat 18, the fluid 20 leakage may be prevented, even when a high air pressure is provided through the microfluidic channel 16.

Also, when the elastic film 13 normally contacts the valve seat 18, if the elastic film 13 contacts the valve seat 18 for a long time, the elastic film 13 may stick to the upper surface of the valve seat 18 and may not be separated from the upper surface of the valve seat 18 due to a chemical or physical reaction, e.g., bonding, therebetween. Accordingly, if the microfluidic device 10 has not been used for a long time, an initialization operation for separating the elastic film 13 and the valve seat 18 from each other may be required. However, in contrast, in the disclosed microfluidic device 10, since the elastic film 13 does not normally contact the valve seat 18, the initialization operation is not required. Accordingly, the flow of the fluid 20 in the microfluidic device 10 may be efficiently controlled without additional processes.

The microfluidic device 10 may be manufactured by, for example, forming a plurality of grooves on opposing surfaces of the first and second substrates 11 and 12, embodiments of which may be formed of glass, plastic or other similar materials. According to the positions and shapes of the grooves formed in the facing surfaces of the first and second substrates 11 and 12, the grooves may function as the reaction chambers 14, the microfluidic channels 16, and the valve seats 18. Also, the holes 15 may be formed by penetrating from non-opposing surfaces of the first and second substrates 11 and 12 into the microfluidic channels 16. As the elastic film 13 is interposed between, and is bonded to, the first and second substrates 11 and 12 in which the reaction chambers 14, the microfluidic channels 16, the valve seats 18, and the holes 15 are formed, the microfluidic device 10 may be completely manufactured. From among various methods of forming the reaction chambers 14, the microfluidic channels 16, and the valve seats 18 in the opposing surfaces of the first and second substrates 11 and 12, a wet etching method will be described below. However, the wet etching method is described as one embodiment, and alternative methods of forming the various components of the microfluidic device 10 may also be used.

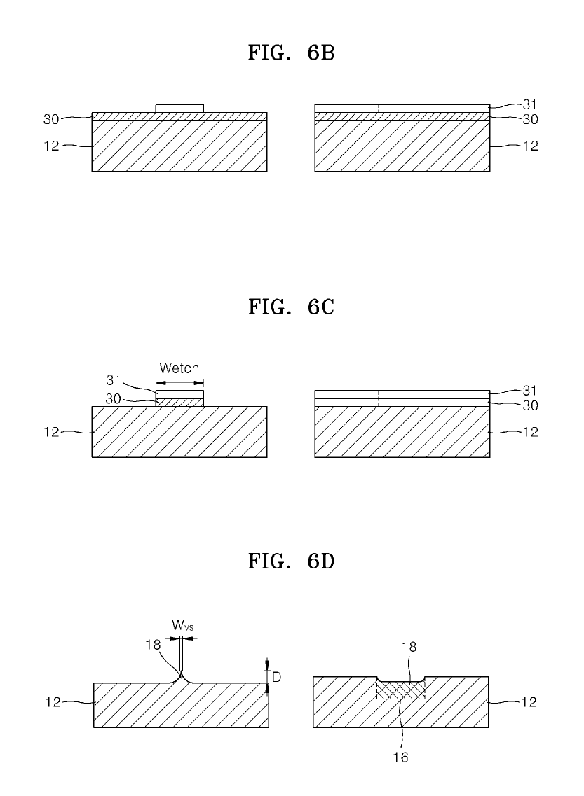

FIGS. 6A through 6D are cross-sectional views illustrating an embodiment of a method of manufacturing the microvalve 17 illustrated in FIGS. 3, 4A, and 4B, according to the present disclosure. In particular, FIGS. 6A through 6D shows a process of forming the valve seat 18. In FIGS. 6A through 6D, left portions are cross-sections taken along line A-A′ of FIG. 3 and right portions are cross-sections taken along line B-B′ of FIG. 3. A wet etching method of forming the valve seat 18 will now be described with reference to FIGS. 6A through 6D.

Initially, referring to FIG. 6A, an etching mask 30 and a photoresist 31 are sequentially coated on a surface of the second substrate 12, in which the valve seat 18 is to be formed. For example, if the second substrate 12 is formed of glass, an embodiment of the etching mask 30 may be formed of polycrystalline silicon (poly-Si).

Then, referring to FIG. 6B, according to a generally used photolithography method, the photoresist 31 may be patterned by exposing and developing the photoresist 31. As such, as illustrated in the left portion of FIG. 6B, in the cross-section taken along line A-A′, a portion of the photoresist 31 for forming the valve seat 18 remains on only a center portion of the etching mask 30. On the other hand, as illustrated in the right portion of FIG. 6B, in the cross-section taken along line B-B′, the portion of the photoresist 31 for forming the valve seat 18 remains across the etching mask 30 corresponding to this portion of the microfluidic device 10. Although the portion of the photoresist 31 for forming only the valve seat 18 is illustrated in FIG. 6B, overall, embodiments include configurations wherein the photoresist 31 is also patterned on the etching mask 30 to form other elements such as the reaction chambers 14 and the microfluidic channels 16. For example, in the right portion of FIG. 6B, dashed lines are illustrated in the photoresist 31. The dashed lines represent that, for example, the photoresist 31 is removed to form the microfluidic channel 16 in front of and at the back of the valve seat 18.

After that, as illustrated in FIG. 6C, the etching mask 30 is patterned into the same pattern as the photoresist 31. For example, in one embodiment, the etching mask 30 may be patterned by removing portions of the etching mask 30, from which the photoresist 31 is removed, using a deep reactive ion etching (“DRIE”) method. As such, as illustrated in the left portion of FIG. 6C, in the cross-section taken along line A-A′, a portion of the etching mask 30 for forming the valve seat 18 remains on only a center portion of the second substrate 12. On the other hand, as illustrated in the right portion of FIG. 6C, in the cross-section taken along line B-B′, the portion of the etching mask 30 for forming the valve seat 18 remains across the second substrate 12. Although only the portion of the etching mask 30 for forming the valve seat 18 is illustrated in FIG. 6C, in one embodiment, overall, the etching mask 30 is also patterned to form other elements such as the reaction chambers 14 and the microfluidic channels 16. For example, in one embodiment, in the right portion of FIG. 6C, dashed lines are illustrated to represent that the etching mask 30 is removed to form the microfluidic channel 16 in front of and at the back of the valve seat 18.

Lastly, referring to FIG. 6D, according to a general wet etching method, the second substrate 12 is etched. In such an embodiment, for example, a hydrofluoric acid (“HF”) solution may be used as an etchant. As such, as illustrated in FIG. 6D, the microfluidic channel 16 and the valve seat 18 may be formed together on the upper surface of the second substrate 12. The left portion of FIG. 6D shows a cross-section of the valve seat 18 taken along line A-A′, and the right portion of FIG. 6D shows a cross-section of the valve seat 18 taken along line B-B′. In the right portion of FIG. 6D, the microfluidic channel 16 formed in front of and at the back of the valve seat 18 is represented using a dashed line.

In the right portion of FIG. 6D, the height of the upper surface of the ultimately formed valve seat 18 is lower than the height of the upper surface of the second substrate 12. In order to arrive at this configuration, a width W_{etch} of the etching mask 30 in the left portion of FIG. 6C is appropriately controlled using general characteristics of wet etching. In general, wet etching refers to isotropic etching in which all crystalline surfaces have substantially the same etching speed and thus an etched portion has a relatively round cross-section instead of a sharp vertical cross-section. Accordingly, the width of a portion that remains when the etching mask 30 is etched is gradually increased in a downward direction, i.e., towards the bottom of the second substrate 12. Also, the width of an upper surface of the remaining portion is reduced as an etching depth is increased. If a substrate to be etched is a glass substrate and an etching mask is formed of poly-Si, various characteristics of a wet etching process may be generally represented by Equation 1.

W_{GLASS}=W_{ETCH}−2×D_{GLASS} [Equation 1]

In Equation 1, W_{GLASS} represents a width of an upper surface of a portion that remains when the glass substrate is etched, W_{ETCH} represents a width of the etching mask, and D_{GLASS} represents an etching depth of the glass substrate. The above correlation may also be applied to the width of the portion of the etching mask 30 for forming the valve seat 18. For example, if the width of the upper surface of the valve seat 18 is at the same height as the upper surface of the second substrate 12 is Wᵥₛ, in order to make the height of the upper surface of the ultimately formed valve seat 18 lower than the height of the upper surface of the second substrate 12, the width Wᵥₛ may have a value equal to or less than a value 0. Accordingly, if an etching depth of the second substrate 12, i.e., the height of the microfluidic channel 16, is 100 μm and the width Wᵥₛ has a value 0, a width W_{ETCH} of the portion of the etching mask 30 for forming the valve seat 18 may be equal to or less than 200 μm. In brief, in order to obtain the width W_{ETCH} of the etching mask 30 to make the height of the upper surface of the valve seat 18 lower than the height of the upper surface of the second substrate 12, a correlation between the width W_{ETCH} of the etching mask 30 and an etching depth D of the second substrate 12 may be represented by Inequality 2.

W_{ETCH}≦2×D [Inequality 2]

For example, the height of the upper surface of the valve seat 18 may be lower than the height of the upper surface of the second substrate 12 by about 0 μm to about 20 μm.

Meanwhile, although not shown in FIGS. 6A through 6D, the etching mask 30 and the photoresist 31 may also be coated and patterned on a surface of the first substrate 11, and then the reaction chambers 14, the microfluidic channels 16, and the empty spaces 19 may be formed on the surface of the first substrate 11 by using a wet etching method.

After the reaction chambers 14, the microfluidic channels 16, the valve seats 18, the empty spaces 19, and the holes 15 are formed in the first and second substrates 11 and 12 as described above, the elastic film 13 is interposed between, and is bonded to, the first and second substrates 11 and 12. For example, an embodiment of a bonding method is described below. Initially, the elastic film 13 is interposed between the first and second substrates 11 and 12. After that, the first and second substrates 11 and 12 and the elastic film 13 are processed using oxygen (O₂) plasma and then are heated in an oven to about 90° C., thereby completely bonding the first and second substrates 11 and 12 to the elastic film 13.

It should be understood that the embodiments described herein should be considered in a descriptive sense only and not for purposes of limitation. Descriptions of features or aspects within each embodiment should typically be considered as available for other similar features or aspects in other embodiments.

What is claimed is:

1. A microfluidic device comprising:
a first substrate; and
a second substrate disposed substantially opposite to the first substrate;
an elastic film interposed between the first substrate and the second substrate;
a microfluidic channel formed in a first surface of the second substrate, wherein the first surface of the second substrate faces the first substrate;
a valve seat which protrudes from the microfluidic channel of the second substrate; and
an empty space formed in a first surface of the first substrate, wherein the first surface of the first substrate faces the second substrate and wherein the empty space corresponds to the valve seat,
wherein an upper surface of the valve seat contacts the elastic film when a controlling air pressure is provided into the empty space, and a gap is formed between the upper surface of the valve seat and the elastic film when a controlling air pressure is not provided into the empty space.

2. The microfluidic device of claim 1, wherein a default setting of the elastic film and the valve seat is a normally open-type microvalve.

3. The microfluidic device of claim 1, wherein the microfluidic channel is recessed in the first surface of the second substrate.

4. The microfluidic device of claim 3, wherein the upper surface of the valve seat formed in the recessed microfluidic channel is lower than the first surface of the second substrate.

5. The microfluidic device of claim 1, wherein the elastic film comprises polydimethylsiloxane.

6. The microfluidic device of claim 1, wherein the first substrate and second substrate comprise at least one of glass and plastic.

7. The microfluidic device of claim 1, further comprising a plurality of reaction chambers formed in at least one of the first surface of the first substrate and the first surface of the second substrate.

8. The microfluidic device of claim 1, further comprising a first hole formed in a second surface of the first substrate which is substantially opposite to the first surface of the first substrate, wherein the first hole is connected to the empty space.

9. The microfluidic device of claim 1, further comprising a second hole formed in a second surface of the second substrate which is substantially opposite to the first surface of the second substrate, wherein the first hole is connected to the microfluidic channel.

10. The microfluidic device of claim 1, further comprising a microfluidic channel formed in the first surface of the first substrate.

11. The microfluidic device of claim 1, wherein the gap between the upper surface of the valve seat and the elastic film when a controlling air pressure is not provided into the empty space is about 0 μm to about 20 μm.

12. A method of manufacturing a microfluidic device, the method comprising:
wet etching a first surface of a first substrate to form an empty space in the first surface of the first substrate;
wet etching a first surface of a second substrate to form a microfluidic channel and a valve seat which protrudes from the microfluidic channel in the first surface of the second substrate;
interposing an elastic film between the first substrate and the second substrate while the first surface of the first substrate and the first surface of the second substrate face each other; and
bonding the elastic film to the first substrate and the second substrate,
wherein the empty space of the first substrate corresponds to the valve seat of the second substrate, and
wherein an upper surface of the valve seat contacts the elastic film when a controlling air pressure is provided into the empty space, and a gap is formed between the upper surface of the valve seat and the elastic film when a controlling air pressure is not provided into the empty space.

13. The method of claim 12, wherein the wet etching a first surface of the second substrate to form the valve seat comprises:
sequentially coating an etching mask and a photoresist on the first surface of the second substrate;
patterning the etching mask and the photoresist until only a portion of the etching mask remains; and
partially etching the first surface of the second substrate such that the upper surface of the valve seat is lower than the first surface of the second substrate.

14. The method of claim 13, wherein the patterning of the etching mask and the photoresist comprises:
patterning the photoresist by exposing and developing the photoresist; and
patterning the etching mask by removing portions of the etching mask from which the photoresist is removed, using a deep reactive ion etching method.

15. The method of claim 13, wherein, when a width of the portion of the etching mask for forming the valve seat is W_{ETCH} and an etching depth of the first surface of the second substrate is D, the following inequality is upheld: W_{ETCH}≦2×D.

16. The method of claim 13, wherein a height of the upper surface of the valve seat is lower than a height of the first surface of the second substrate by about 0 μm to about 20 μm.

17. The method of claim 12, wherein the interposing of the elastic film between the first substrate and the second substrate and the bonding of the elastic film to the first substrate and the second substrate comprises:
interposing the elastic film between the first surface of the first substrate and the first surface of the second substrate;
processing the first substrate, the second substrate and the elastic film using oxygen plasma; and
heating the first substrate, the second substrate and the elastic film in an oven.
